(19) United States
(12) Patent Application Publication
(10) Pub. No.: US 2009/0226882 A1
(43) Pub. Date: Sep. 10, 2009

(54) Method for Identifying Fungicidally Active Compounds that are Based on Ipp Isomerases

(75) Inventors: Peter Schreier, Köln (DE); Birgitta Leuthner, Langenfeld (DE); Thorsten Leicher, Langenfeld (DE)

Correspondence Address: WOMBLE CARLYLE SANDRIDGE & RICE, PLLC, ATTN: PATENT DOCKETING, P.O. BOX 7037, ATLANTA, GA, 30357-0037, US

(73) Assignee: Bayer Cropscience AG, Monheim (DE)

(21) Appl. No.: 11/914,907

(22) PCT Filed: May 19, 2006
(86) PCT No.: PCT/EP2006/004758

§ 371 Date: Jul. 7, 2008

(30) Foreign Application Priority Data
Jun. 1, 2005 (DE) 102005025191.9

(51) Int. Cl. C12Q 1/533; C12N 15/31; C12N 15/63; C12N 9/90; C12N 1/21; C12N 1/19; C12N 5/10; A01P 3/00

(52) U.S. Cl. 435/4; 536/23.74; 435/320.1; 435/233; 435/252.33; 435/254.21; 435/254.23; 435/348; 435/419

(57) ABSTRACT

The invention relates to a method for identifying fungicides, to the use of fungal IPP isomerase for identifying fungicides, and to the use of inhibitors of the IPP isomerase as fungicides.

The invention relates to a method for identifying fungicides, to the use of fungal isopentenyl pyrophosphate isomerase for identifying fungicides, and to the use of inhibitors of said isopentenyl pyrophosphate isomerase as fungicides.

Undesired fungal growth which leads every year to considerable damage in agriculture can be controlled by the use of fungicides. The demands made on fungicides have increased constantly with regard to their activity, costs and, above all, ecological soundness. There exists therefore a demand for new substances or classes of substances which can be developed into potent and ecologically sound new fungicides. In general, it is customary to search for such new lead structures in greenhouse tests. However, such tests require a high input of labor and a high financial input. The number of the substances which can be tested in the greenhouse is, accordingly, limited. An alternative to such tests is the use of what are known as high-throughput screening (HTS) methods. This involves testing a large number of individual substances with regard to their effect on cells, individual gene products or genes in an automated method. When certain substances are found to have an effect, they can be studied in conventional screening methods and, if appropriate, developed further.

Advantageous targets for fungicides are frequently searched for in essential biosynthetic pathways. Ideal fungicides are, moreover, those substances which inhibit gene products which have a decisive importance in the manifestation of the pathogenicity of a fungus.

It was therefore an aim of the present invention to identify, and make available, a suitable new target for potential fungicidal active compounds and to provide a method which makes possible the identification of modulators of this target which can be used as fungicides.

Isopentenyl pyrophosphate isomerase (EC 5.3.3.2), also known as isopentenyl pyrophosphate Δ-isomerase, isopentenyl diphosphate Δ-isomerase, or methylbutenyl pyrophosphate isomerase, catalyzes the isomerization of the carbon double bond of isopentenyl pyrophosphate (IPP), producing dimethylallyl pyrophosphate (DMAPP) (FIG. 1).

Isopentenyl pyrophosphate isomerase—also abbreviated as IPP isomerase or IPPI hereinbelow—thus catalyzes an essential step of isoprenoid biosynthesis with more than 23 000 known metabolites. The synthetic pathway has been described in all organisms, providing different substance classes. These include the sterols, the carotenoids, the dolichols, the ubiquinones and prenylated proteins. IPP isomerase catalyzes the critical activation step in the synthetic pathway, which converts isopentenyl pyrophosphate (IPP) to the strongly electrophilic isomer, dimethylallyl diphosphate (DMAPP). IPP and DMAPP are substrates for -prenyl transferases which synthesize polyisoprenoid chains.

The corresponding gene, IDI1, was shown to be essential in S. cerevisiae (an ascomycete) and occurs only once in the genome (Mayer et al. 1992).

The isoprenoid metabolic pathway is present in all organisms and generates a multiplicity of small, usually lipophilic substances which carry out a number of important functions. A prominent part is played here by the sterols, components of eukaryotic membranes and hormones, the carotenes, photoreceptors for seeing and in photosynthesis, coenzymes in respiration, moulting hormones in insects, and the cytokinins, hormones in plants. Isoprenoids are synthesized in two different phases. The first phase comprises stepwise synthesis of hydroxymethylglutaryl coenzyme A from three molecules of acetyl-CoA. This is reduced by HMG-CoA reductase to give mevalonate and is then fused in a number of further reactions to give squalene. A center point of this first phase is IPP isomerase which provides both isopentenyl pyrophosphate and dimethylallyl pyrophosphate (Wouters et al., 2003). These two compounds are further fused in a head-to-tail reaction to give geranyl diphosphate. The latter may then be metabolically processed further to give different products, depending on the organism. In the case of fungi, the synthesis of sterols is of essential importance here.

IPP isomerase has already been disclosed for a number of fungi (for this, see also FIG. 2). These include, for example, S. cerevisiae, S. pombe etc.

IPP isomerases are characterized by specific motifs at the amino acid level and may be identified inter alia on the basis of said motifs. Thus it was demonstrated by specific mutagenesis in yeast that two amino acids have essential importance. These amino acids are Cys and Gln in positions 139 and 207 (S. cerevisiae, see also FIG. 2), embedded in the sequences CCSH and HEIDY, respectively (Street et al. 1994, Wouters et al, 2003).

IPP isomerase genes have been cloned from various organisms, including also various yeasts such as Saccharomyces cerevisiae (Swissprot Accession No.: P15496), Schizosaccharomyces pombe (Swissprot Accession No.: Q10132), and Phaffia rhodozyma (Swissprot Accession No.: O42641). The sequence similarities are significant within the eukaryotic classes.

It was furthermore the object of the present invention to identify new targets of fungicides in fungi, in particular in phytopathogenic fungi, and to make available a method in which inhibitors of such a target or polypeptide can be identified and tested for their fungicidal properties. It was therefore the object of the present invention to test whether IPP isomerase of the plant-pathogenic basidiomycete U. maydis is also an essential gene and its removal leads to nonviable spores, and whether IPP isomerase of plant-pathogenic fungi is a suitable target for fungicides in principle.

The object was achieved by isolating from a phytopathogenic fungus, U. maydis, the nucleic acid coding for IPP isomerase (ipi1), obtaining the polypeptide encoded thereby (IPI1) and providing a method which can be used for determining inhibitors of said enzyme. The inhibitors identified by said method may actually be used against fungi in vivo.

### DESCRIPTION OF THE FIGURES

FIG. 1: Diagrammatic representation of the isomerization of isopentenyl pyrophosphate to dimethylallyl pyrophosphate, catalyzed by IPP isomerase.

FIG. 2: Comparison of disclosed IPP isomerase proteins from fungi and phytopathogenic fungi (UM=U. maydis; PC=P. chrysosporium; IDI1=S. cerevisiae; ADL=A. gossypii; Idi1=S. pombe; MG=M. grisea; CA=C. albicans; AN=A. nidulans; FG=F. graminis; NC=N. crassa; PHYSO=P. soyae; PHYTRA=P. ramorum). Regions with identity or high homology are highlighted in gray.

FIG. 3: 12% bis-Tris-SDS gel for depicting heterologous expression of U. maydis IPP isomerase in E. coli.
1+18=marker; 2-13=eluted fractions with 250 mM imidazole; 14-17=eluted fractions with 1 M imidazole; 19=cytoplasmic fraction; 20=membrane fraction; 21=flow through; 22+23=1st and 2nd wash fractions; 24=pooled fractions Nos. 6, 7, 8, 9

FIG. 4: Spore analysis of ipi knock out strains. Candidate spores were streaked out on medium (PD medium) without (1A-4A, PD/Hyg medium) and with selection, 1B-4B. If the switched-off gene is essential, no spores should grow on PD/Hyg medium, if all spores are haploid. It happens again and again that diploid spores are selected as candidates. Therefore, in all cases in which spores grew on PD/Hyg medium, they were examined with the aid of a PCR analysis. Said spores were shown to be diploid, i.e. they still contained a copy of the ipi wild type gene.

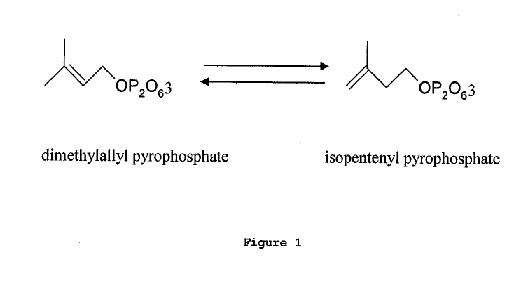

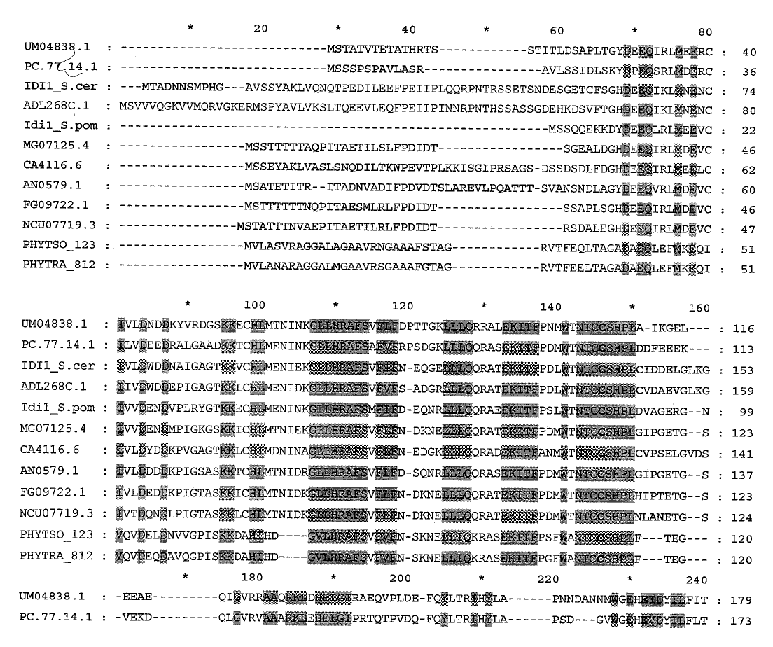

SEQ ID NO:1 Nucleic acid sequence coding for Ustilago maydis isopentenyl pyrophosphate isomerase.
SEQ ID NO: 2 Amino acid sequence of Ustilago maydis isopentenyl pyrophosphate isomerase.

### DEFINITIONS

The term “homology” or “identity” is intended to mean the number of corresponding amino acids (identity) with other proteins, expressed in percent. Preference is given to determining said identity by comparing a given sequence to other proteins with the aid of computer programs. If sequences that are compared to one another have different lengths, identity must be determined in such a way that the number of amino acids common to both the shorter sequence and the longer sequence determines the percentage identity. Identity may be determined routinely by means of known and publicly available computer programs such as, for example, ClustalW (Thompson et al., Nucleic Acids Research 22 (1994), 4673-4680). ClustalW, for example, is made publicly available by Julie Thompson (Thompson@EMBL-Heidelberg.DE) and Toby Gibson (Gibson@EMBL-Heidelberg.DE), European Molecular Biology Laboratory, Meyerhofstrasse 1, D 69117 Heidelberg, Germany. ClustalW may likewise be downloaded from various Internet pages, for example at IGBMC (Institut de Génétique et de Biologie Moléculaire et Cellulaire, B.P.163, 67404 Illkirch Cedex, France; ftp://ftp-igbmc.u-strasbg.fr/pub/) and at EBI (ftp://ftp.ebi.ac.uk/pub/software/) and also on all mirrored EBI Internet pages (European Bioinformatics Institute, Wellcome Trust Genome Campus, Hinxton, Cambridge CB10 1SD, UK). When using version 1.8 of the ClustalW computer program in order to determine identity, for example, between a given reference protein and other proteins, the following parameters must be set: KTUPLE=1, TOPDIAG=5, WINDOW=5, PAIRGAP=3, GAPOPEN=10, GAPEXTEND=0.05, GAPDIST=8, MAXDIV=40, MATRIX=GONNET, ENDGAPS(OFF), NOPGAP, NOHGAP. One possibility of finding similar sequences is to carry out sequence database searches. This involves defining one or more sequences as “query”. This query sequence is then compared with sequences present in the selected databases by means of statistical computer programs. Such database queries (“blast searches”) are known to the skilled worker and may be carried out at various providers. If, for example, such a database query is carried out at NCBI (National Center for Biotechnology Information, http://www.ncbi.nlm.nih.gov/), the standard settings defined for the particular comparative query should be used. For protein sequence comparisons (“blastp”), these settings are as follows: limit entrez=not activated; filter=low complexity activated; expect value=10; word size=3; matrix=BLOSUM62; gap costs: existence=11, extension=1. Apart from other parameters, the proportion of identity between the query sequence and the similar sequences found in the databases is also depicted as result of such a query. A protein of the invention is therefore intended to mean in connection with the present invention those proteins which, with the use of at least one of the above-described methods for determining identity, are at least 70%, preferably at least 75%, particularly preferably at least 80%, more preferably at least 85%, and in particular at least 90%, identical.

The term “complete IPP isomerase”, as used herein, describes an IPP isomerase encoded by a complete coding region of a transcriptional unit comprising an ATG start codon and comprising all information-carrying exon regions of the IPP isomerase-encoding gene present in the source organism, and the signals required for correct termination of transcription.

The term “biological activity of an IPP isomerase”, as used herein, refers to the ability of a polypeptide to catalyze the above-described reaction, i.e. isomerization of the carbon double bond of isopentenyl pyrophosphate and dimethylallyl pyrophosphate.

The term “active fragment”, as used herein, describes IPP isomerase-encoding nucleic acids which are no longer complete but which still code for polypeptides having the biological activity of an IPP isomerase, which polypeptides are capable of catalyzing a reaction characteristic of IPP isomerase, as described above. Such fragments are shorter than the above-described complete, IPP isomerase-encoding nucleic acids. In this context, nucleic acids may have been removed both at the 3′ and/or 5′ ends of the sequence, or else parts of the sequence which do not have a decisive adverse effect on the biological activity of IPP isomerase may have been deleted, i.e. removed. A lower or else, where appropriate, an increased activity which nevertheless still allows the resulting IPP isomerase fragment to be characterized or used is considered here as sufficient for the purposes of the term as used herein. The term “active fragment” may likewise refer to the amino acid sequence of IPP isomerase and, in this case, applies analogously to the comments made above on those polypeptides which, compared to the above-defined complete sequence, no longer contain certain parts, with the biological activity of the enzyme not being adversely affected in any decisive way, however. The fragments here may have different lengths.

The terms “IPP isomerase inhibition assay” or “inhibition assay”, as used herein, refer to a method or an assay which allows inhibition of the enzymic activity of a polypeptide having the activity of an IPP isomerase by one or more chemical compounds (candidate compound(s)) to be detected, enabling said chemical compound to be identified as IPP isomerase inhibitor.

The term “gene”, as used herein, is the name for a section from the genome of a cell, which is responsible for the synthesis of a polypeptide chain.

The term “fungicide” or “fungicidal”, as used herein, refers to chemical compounds which are suitable for controlling human-, animal- and plant-pathogenic fungi, in particular plant-pathogenic fungi. Such plant-pathogenic fungi are listed below, with the list not being final: Plasmodiophoromycetes, oomycetes, chytridiomycetes, zygomycetes, ascomycetes, basidiomycetes and deuteromycetes, for example

Pythium species such as, for example, Pythium ultimum, Phytophthora species such as, for example, Phytophthora infestans, Pseudoperonospora species such as, for example, Pseudoperonospora humuli or Pseudoperonospora cubensis, Plasmopara species such as, for example, Plasmopara viticola, Bremia species such as, for example, Bremia lactucae, Peronospora species such as, for example, Peronospora pisi or P. brassicae, Erysiphe species such as, for example, Erysiphe graminis, Sphaerotheca species such as, for example, Sphaerotheca fuliginea, Podosphaera species such as, for example, Podosphaera leucotricha, Venturia species such as, for example, Venturia inaequalis, Pyrenophora species such as, for example, Pyrenophora teres or P. graminea (conidial form: Drechslera, syn: Helminthosporium), Cochliobolus species such as, for example, Cochliobolus sativus (conidial form: Drechslera, syn: Helminthosporium), Uromyces species such as, for example, Uromyces appendiculatus, Puccinia species such as, for example, Puccinia recondita, Sclerotinia species such as, for example, Sclerotinia sclerotiorum, Tilletia species such as, for example, Tilletia caries; Ustilago species such as, for example, Ustilago nuda or Ustilago avenae, Pellicularia species such as, for example, Pellicularia sasakii, Pyricularia species such as, for example, Pyricularia oryzae, Fusarium species such as, for example, Fusarium culmorum, Botrytis species, Septoria species such as, for example, Septoria nodorum, Leptosphaeria species such as, for example, Leptosphaeria nodorum, Cercospora species such as, for example, Cercospora canescens, Alternaria species such as, for example, Alternaria brassicae or Pseudocercosporella species such as, for example, Pseudocercosporella herpotrichoides.

Other examples of particular interest are Magnaporthe grisea, Cochliobulus heterostrophus, Nectria hematococcus and Phytophtora species.

Fungicidally active compounds found with the aid of the IPP isomerases of the invention from plant-pathogenic fungi may also interact with IPP isomerase from human-pathogenic fungal species, however, the interaction with the different IPP isomerases present in these fungi not necessarily always being equally strong.

The present inventions therefore also relate to the use of inhibitors of IPP isomerase for preparing remedies for the treatment of diseases caused by human-pathogenic fungi.

In this context, the following human-pathogenic fungi which may cause the pathologies listed below are of particular interest:

Dermatophytes such as, for example, Trichophyton spec., Microsporum spec., Epidermophyton floccosum or Keratomyces ajelloi, which cause, for example, foot mycoses (tinea pedis),

Yeasts such as, for example, Candida albicans, which causes candidal esophagitis and dermatitis, Candida glabrata, Candida krusei or Cryptococcus neoformans, which may cause, for example, pulmonal cryptococcosis or else torulosis,

Molds such as, for example, Aspergillus fumigatus, A. flavus, A. niger, which cause, for example, bronchopulmonary Aspergillosis or fungal sepsis, Mucor spec., Absidia spec., or Rhizopus spec., which cause, for example, zygomycoses (intravasal mycoses), Rhinosporidium seeberi, which causes, for example, chronic granulomatous pharyngitis and tracheitis, Madurella myzetomatis, which causes, for example, subcutaneous mycetomas, Histoplasma capsulatum, which causes, for example, reticuloendothelial cytomycosis and Darling's disease, Coccidioides immitis, which causes, for example, pulmonary coccidioidomycosis and sepsis, Paracoccidioides brasiliensis, which causes, for example, South American blastomycosis, Blastomyces dermatitidis, which causes, for example, Gilchrist's disease and North American blastomycosis, Loboa loboi, which causes, for example, keloid blastomycosis and Lobo's disease, and Sporothrix schenckii, which causes, for example, sporotrichosis (granulomatous dermal mycosis).

Fungicidally active compounds which are found with the aid of an IPP isomerase obtained from a particular fungus, in this case from Ustilago maydis, may therefore also interact with IPP isomerase from numerous other fungal species, especially also with plant-pathogenic fungi, said interaction with the different IPP isomerases present in these fungi not necessarily always being equally strong. This explains inter alia the observed selectivity of the substances acting on this enzyme.

The term “homologous promoter”, as used herein, refers to a promoter which controls expression of the gene in question in the source organism. The term “heterologous promoter” as used herein, refers to a promoter which has properties different from those of that promoter which controls expression of the gene in question in the source organism.

The term “competitor”, as used herein, refers to the property of the compounds of competing with other compounds, optionally to be identified, for binding to IPP isomerase and of displacing these compounds from the enzyme or being displaced thereby.

The term “inhibitor” or “specific inhibitor”, as used herein, refers to a substance which directly inhibits an enzymic activity of IPP isomerase. Such an inhibitor is preferably “specific”, i.e. it inhibits specifically IPP isomerase activity at a concentration lower than the concentration of an inhibitor required for causing a different effect not related thereto. Said concentration is preferably lower by a factor of two, particularly preferably by a factor of five and very particularly preferably by at least a factor of ten or a factor of 20, than the concentration of a compound required for causing an unspecific effect.

The term “modulator”, as used herein, represents a generic term for inhibitors and activators. Modulators may be small organochemical molecules, peptides or antibodies that bind to the polypeptides of the invention or influence their activity. Furthermore, modulators may be small organochemical molecules, peptides or antibodies that bind to a molecule which in turn binds to the polypeptides of the invention, thereby influencing their biological activity. Modulators may be natural substrates and ligands or may be structural or functional mimetics thereof. However, preference is given to the term “modulator”, as used herein, being those molecules which are not the natural substrates or ligands.

### DESCRIPTION OF THE INVENTION

The present invention, for the first time, makes available the complete sequence of an IPP isomerase from the plant-pathogenic fungus, Ustilago maydis, which sequence enables IPP isomerases, in particular from plant-pathogenic fungi, to be explored further and thereby a new target protein for identifying novel fungicidally active compounds to be made accessible.

Previously, research on IPP isomerase has been limited primarily to its pharmacological importance (Cheng and Oldfield, 2004; Thompsom et al., 2002; Rohdich et al. 2004). This includes nevertheless work on inhibitors of this enzyme, which are intended for pharmaceutical application. Inhibitors of IPP isomerase, such as natural inhibitors or analogs of the transitional state of the substrate of IPP isomerase, have been described (Wouters et al., 2003).

Despite extensive research on IPP isomerase, the enzyme has not been known previously to be a possible target protein of fungicidally active substances in fungi. The present invention therefore, for the first time, demonstrates that IPP isomerase is an important enzyme, particularly to fungi, and is therefore particularly suited to be used as a target protein for the search for further and improved fungicidally active compounds.

IPP isomerase inhibitors having fungicidal action have not been described previously. Although the enzyme is known to be essential in S. cerevisiae (Mayer et al. 1992), none of the applications discusses the question, whether the fungal IPP isomerase enzyme can be influenced, for example inhibited, by active compounds, in particular in plant-pathogenic fungi, and whether fungi, in particular plant-pathogenic fungi, can be controlled in vivo by an IPP isomerase-modulating active compound. Thus IPP isomerase has not been described as target protein for fungicides previously. There are no known active compounds that have fungicidal action and whose site of action is IPP isomerase.

Within the framework of the present invention, IPP isomerase has now been shown to be a possible point of attack or target for fungicidal active compounds in plant-pathogenic fungi, i.e. inhibition of IPP isomerase could result in the fungus being damaged or killed. Thus an IPP isomerase-encoding gene according to SEQ ID NO:1 (ipi1) was identified in the plant-pathogenic fungus, Ustilago maydis. Knocking out this gene proved to be lethal. No viable knock-out spores of U. maydis were obtained. In further experiments aimed at the accessibility of IPP isomerase to active compounds in vitro and also in vivo, the IPP isomerase enzyme was also established as being a polypeptide which may be used for identifying modulators or inhibitors of its enzymic activity in suitable assays, which is not obvious with various theoretically interesting targets.

The present invention therefore involved developing a method suitable for determining IPP isomerase activity and inhibition of said activity in an inhibition assay, identifying in this way inhibitors of the enzyme, for example in HTS and UHTS methods, and testing their fungicidal properties. The present invention also demonstrated that inhibitors of IPP isomerase from fungi can be used as fungicides.

It was also found within the framework of the present invention that IPP isomerase can also be inhibited in vivo by active compounds and that a fungal organism treated with said compounds can be damaged and killed by treatment with said compounds. The inhibitors of a fungal IPP isomerase can thus be used as fungicides in crop protection or as antimycotics in pharmacological indications. For example, the present invention shows that inhibition of IPP isomerase by any substances identified in a method of the invention results in the death of the treated fungi in synthetic media or on the plant.

IPP isomerase may be obtained from various plant-pathogenic or else human- or animal-pathogenic fungi, for example from fungi such as the plant-pathogenic fungus, U. maydis. Fungal IPP isomerase may be prepared by expressing the gene, for example, recombinantly in Escherichia coli and preparing an enzyme preparation from E. coli cells (example 1). Preference is given to using IPP isomerases from plant-pathogenic fungi in order to identify fungicides which can be employed in crop protection. If the aim is to identify fungicides or antimycotics to be used in pharmacological indications, the use of IPP isomerases from human- or animal-pathogenic fungi is recommended.

To express the ipi1-encoded U. maydis polypeptide IPI1, the corresponding ORF was thus amplified by means of PCR via selected primers according to methods known to the skilled worker. The corresponding DNA was cloned into the pET21b expression vector, so that the IPI1 protein is expressed with a His₆ tag. IPI1 was expressed by transforming the plasmid into E. coli BL21(DE3), and the polypeptide was obtained according to example 1.

The present invention therefore also provides a complete genomic sequence of a plant-pathogenic fungus coding for an IPP isomerase and describes the use thereof or the use of the polypeptide encoded thereby for identifying inhibitors of said enzyme.

The present invention therefore also relates to the nucleic acid according to SEQ ID NO:1 from the fungus, Ustilago maydis, which nucleic acid codes for a polypeptide having the enzymic function of an IPP isomerase.

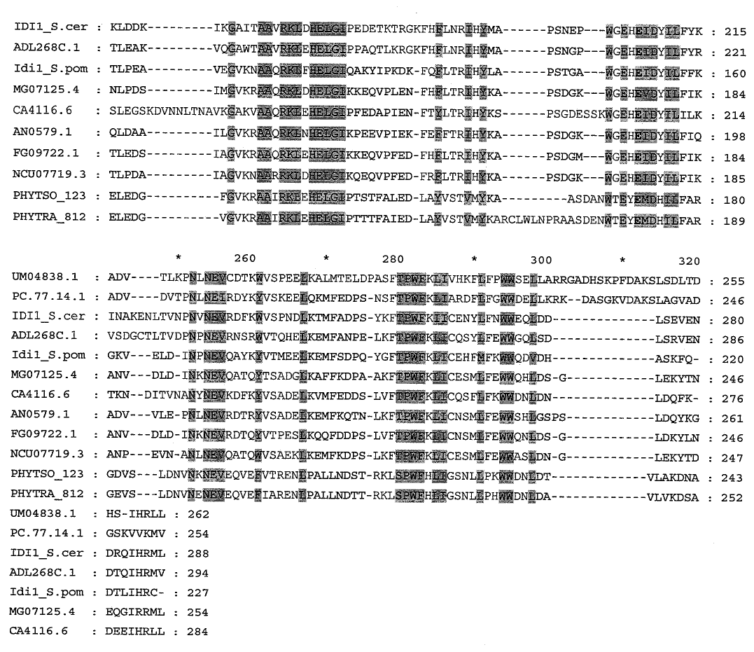

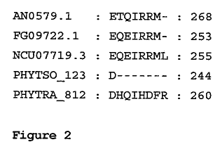

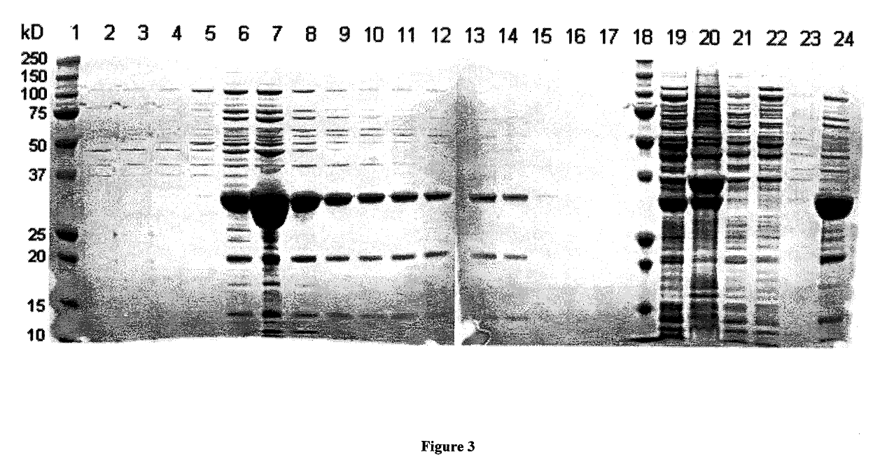

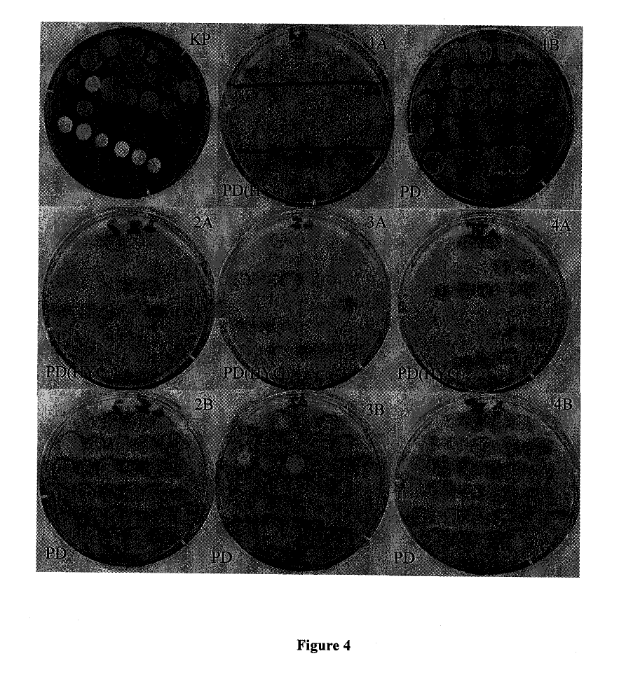

Owing to the homologies (see also FIG. 2) present in species-specific nucleic acids coding for IPP isomerases, it is also possible to identify and use IPP isomerases from other plant-pathogenic fungi in order to achieve the above object, i.e. they may likewise be used for identifying inhibitors of an IPP isomerase, which inhibitors can in turn be used as fungicides in crop protection. However, it is also conceivable to use a different fungus which is not pathogenic to plants, or its IPP isomerase or the sequence coding therefor, in order to identify fungicidal inhibitors of IPP isomerase. Owing to the sequence set forth herein in SEQ ID NO:1 and to primers possibly derived therefrom and also, where appropriate, with the aid of the consensus sequence sections depicted in FIG. 2, in particular the abovementioned (amino acid) sequence sections “CCSH” and “HEIDY”, it is possible for the skilled worker to obtain and identify, for example, by means of PCR further nucleic acids coding for IPP isomerases from other (plant-pathogenic) fungi or to classify available nucleic acid or amino acid sequences. Such nucleic acids and their use in methods for identifying fungicidally active compounds are considered as being encompassed by the present invention.

Further IPP isomerase-encoding nucleic acid sequences from other fungi can be identified with the aid of the nucleic acid sequence of the invention and of sequences obtained by the methods described above.

The present invention therefore relates to nucleic acids from plant-pathogenic fungi which code for a polypeptide having the enzymic activity of an IPP isomerase, in particular polypeptides comprising the above-described motif.

Preference is given to subject matter of the present invention being nucleic acids from the plant-pathogenic fungal species listed under definitions above, which nucleic acids code for a polypeptide having the enzymic activity of an IPP isomerase.

The present invention particularly preferably relates to the nucleic acid coding for Ustilago maydis IPP isomerase and having SEQ ID NO:1 and to the nucleic acids coding for the polypeptides according to SEQ ID NO:2 or active fragments thereof.

The nucleic acids of the invention are in particular single-stranded or double-stranded deoxyribonucleic acids (DNA) or ribonucleic acids (RNA). Preferred embodiments are fragments of genomic DNA, and cDNAs.

Particular preference is given to the nucleic acids of the invention comprising a sequence from plant-pathogenic fungi, coding for a polypeptide having the enzymic activity of an IPP isomerase, selected from
a) a sequence according to SEQ ID NO: 1,
b) sequences coding for a polypeptide comprising the amino acid sequence according to SEQ ID NO: 2,
c) sequences which hybridize to the sequences defined under a) and b) at a hybridization temperature of 42-65° C.,
d) sequences which are at least 80%, preferably at least 85%, and particularly preferably at least 90%, identical to the sequences defined under a) and b), and
e) sequences which are complementary to the sequences defined under a) to d).

As stated above, the present invention is not limited to only Ustilago maydis IPP isomerase. It is also possible, in an analogous manner known to the skilled worker, to obtain polypeptides having the activity of an IPP isomerase from other fungi, preferably from plant-pathogenic fungi, which can then be employed, for example, in a method of the invention. Preference is given to using Ustilago maydis IPP isomerase.

The present invention furthermore relates to DNA constructs comprising a nucleic acid of the invention and a homologous or heterologous promoter.

The selection of heterologous promoters depends on whether pro- or eukaryotic cells or cell-free systems are used for expression. Examples of heterologous promoters are the 35S promoter of cauliflower mosaic virus for plant cells, the alcohol dehydrogenase promoter for yeast cells, the T3, T7 or SP6 promoters for prokaryotic cells or cell-free systems.

Preference should be given to using fungal expression systems such as, for example, the Pichia pastoris system, transcription here being driven by the methanol-inducible AOX promoter.

The present invention further relates to vectors comprising a nucleic acid of the invention, a regulatory region of the invention or a DNA construct of the invention. Vectors which may be used are any phages, plasmids, phagemids, plasmids, cosmids, YACs, BACs, artificial chromosomes or particles suitable for particle bombardment, all of which are used in molecular-biological laboratories.

Examples of preferred vectors are the p4XXprom vector series (Mumberg et al., 1995) for yeast cells, pSPORT vectors (Life Technologies) for bacterial cells or the Gateway vectors (Life Technologies) for various expression systems in bacterial cells, plants, P. pastoris, S. cerevisiae or insect cells.

The present invention also relates to host cells containing a nucleic acid of the invention, a DNA construct of the invention or a vector of the invention.

The term “host cell”, as used herein, refers to cells which do not naturally contain the nucleic acids of the invention.

Suitable host cells are both prokaryotic cells, preferably E. coli, and eukaryotic cells such as cells of Saccharomyces cerevisiae, Pichia pastoris, insects, plants, frog oocytes and mammalian cell lines.

The present invention furthermore relates to polypeptides having the biological activity of an IPP isomerase which are encoded by the nucleic acids of the invention.

Preference is given to the polypeptides of the invention comprising an amino acid sequence from plant-pathogenic fungi, selected from
(a) the sequence according to SEQ ID NO:2,
(b) sequences which are at least 80%, preferably at least 85%, particularly preferably 90%, and very particularly preferably 95%, identical to the sequence defined under a),
(c) fragments of the sequences listed under a) or b), which have the same biological activity as the sequence defined under a).

The term “polypeptides” as used in the present context refers not only to short amino acid chains which are generally referred to as peptides, oligopeptides or oligomers, but also to longer amino acid chains which are normally referred to as proteins. It comprises amino acid chains which can be modified either by natural processes, such as post-translational processing, or by chemical prior-art methods. Such modifications may occur at various sites and repeatedly in a polypeptide, such as, for example, on the peptide backbone, on the amino acid side chain, on the amino and/or the carboxyl terminus. For example, they comprise acetylations, acylations, ADP ribosylations, amidations, covalent linkages to flavins, heme moieties, nucleotides or nucleotide derivatives, lipids or lipid derivatives or phosphatidylinositol, cyclizations, disulfide bridge formations, demethylations, cystine formations, formylations, gamma-carboxylations, glycosylations, hydroxylations, iodinations, methylations, myristoylations, oxidations, proteolytic processings, phosphorylations, selenoylations and tRNA-mediated amino acid additions.

The polypeptides according to the invention may exist in the form of “mature” proteins or as parts of larger proteins, for example as fusion proteins. They can furthermore exhibit secretion or leader sequences, pro-sequences, sequences which allow simple purification, such as polyhistidine residues, or additional stabilizing amino acids. The proteins according to the invention may also exist in the form in which they are naturally present in their source organism, from which they can be obtained directly, for example. Likewise, active fragments of an IPP isomerase may be employed in the methods according to the invention, as long as they make possible the determination of the enzymic activity of the polypeptide, or its inhibition by a candidate compound.

In comparison with the corresponding regions of naturally occurring IPP isomerases, the polypeptides used in the methods according to the invention can have deletions or amino acid substitutions, as long as they still exhibit at least the biological activity of a complete IPP isomerase. Conservative substitutions are preferred. Such conservative substitutions comprise variations, one amino acid being replaced by another amino acid from the following group:

1. Small, aliphatic residues, which are non-polar or of little polarity: Ala, Ser, Thr, Pro and Gly;
2. Polar, negatively charged residues and their amides: Asp, Asn, Glu and Gln;
3. Polar, positively charged residues: His, Arg and Lys;
4. Large, aliphatic, non-polar residues: Met, Leu, Ile, Val and Cys; and
5. Aromatic residues: Phe, Tyr and Trp.

One possible IPP isomerase purification method is based on preparative electrophoresis, FPLC, HPLC (for example using gel filtration columns, reversed-phase columns or mildly hydrophobic columns), gel filtration, differential precipitation, ion-exchange chromatography or affinity chromatography (cf. Example 2).

A rapid method of isolating the IPP isomerases which are synthesized by host cells starts with expressing a fusion protein, where the fusion partner may be purified in a simple manner by affinity purification. For example, the fusion partner may be an MBP tag. The fusion protein may in this case be purified on amylose resin. The fusion moiety can be removed by partial proteolytic cleavage, for example at linkers between the fusion moiety and the polypeptide according to the invention which is to be purified. The linker can be designed in such a way that it includes target amino acids, such as arginine and lysine residues, which define sites for trypsin cleavage. Standard cloning methods using oligonucleotides may be employed for generating such linkers.

Other purification methods which are possible are based, in turn, on preparative electrophoresis, FPLC, HPLC (e.g. using gel filtration columns, reversed-phase columns or mildly hydrophobic columns), gel filtration, differential precipitation, ion-exchange chromatography and affinity chromatography.

The terms “isolation or purification” as used in the present context mean that the polypeptides according to the invention are separated from other proteins or other macromolecules of the cell or of the tissue. The protein content of a composition containing the polypeptides according to the invention is preferably at least 10 times, more preferably at least 100 times, higher than in a host cell preparation.

The polypeptides according to the invention may also be affinity-purified without fusion moieties with the aid of antibodies which bind to the polypeptides.

The method of preparing polypeptides with the enzymic activity of an IPP isomerase, such as, for example, the polypeptide U. maydis IPI1, is thus characterized in that
(a) a host cell comprising at least one expressible nucleic acid sequence coding for a fungal polypeptide with the biological activity of an IPP isomerase is cultured under conditions which ensure the expression of this nucleic acid, or
(b) an expressible nucleic acid sequence encoding a fungal polypeptide with the biological activity of an IPP isomerase is expressed in an in-vitro system, and
(c) the polypeptide is recovered from the cell, the culture medium or the in-vitro system.

The cells thus obtained which comprise the polypeptide according to the invention, or the purified polypeptide thus obtained, are suitable for use in methods of identifying IPP isomerase modulators or inhibitors.

The present invention also relates to the use of polypeptides from fungi, preferably from plant-pathogenic fungi, which exert at least one biological activity of an IPP isomerase, in methods for identifying fungicides, it being possible for the IPP isomerase inhibitors to be used as fungicides. Particular preference is given to using Ustilago maydis IPP isomerase.

Fungicidal active compounds found with the aid of an IPP isomerase from a particular fungal species and based on a method of the invention may also interact with IPP isomerase from other fungal species, said interaction with the different IPP isomerases present in these fungi not necessarily always being equally strong. This explains inter alia the selectivity of active substances. Utilization as fungicide also in other fungi of the active compounds found by using a specific IPP isomerase may also be attributed to the fact that IPP isomerases of various fungal species are closely related and exhibit a distinct homology over relatively large regions. Thus FIG. 2 reveals that such a homology exists between S. cerevisiae, S. pombe, and U. maydis over substantial sequence sections and that, as a result, the action of the substances found, for example, with the aid of U. maydis IPP isomerase will not be limited to U. maydis.

The present invention therefore also relates to a method for identifying fungicides by assaying potential inhibitors or modulators of the enzymic activity of IPP isomerase (candidate compound or test compound) in an IPP isomerase inhibition assay, it being possible for an inhibitor or modulator of IPP isomerase, which has been found in an activity assay, to be tested subsequently for its efficacy as fungicide in vivo, i.e. on a fungus.

Methods which are suitable for identifying modulators, in particular inhibitors or antagonists, of the polypeptides according to the invention are generally based on the determination of the activity or the biological functionality of the polypeptide. Suitable for this purpose are, in principle, methods based on intact cells (in-vivo methods), but also methods which are based on the use of the polypeptide isolated from the cells, which may be present in purified or partially purified form or else as a crude extract. These cell-free in-vitro methods, like in-vivo methods, can be used on a laboratory scale, but preferably also in HTS or UHTS methods. Following the in-vivo or in-vitro identification of modulators of the polypeptide, fungal cultures can be assayed in order to test the fungicidal activity of the compounds which have been found.

A large number of assay systems for the purpose of assaying compounds and natural extracts are preferably designed for high throughput numbers in order to maximize the number of substances assayed within a given period. Assay systems based on cell-free processes require purified or semipurified protein. They are suitable for an “initial” assay, which aims mainly at detecting a possible effect of a substance on the target protein. Once such an initial assay has taken place, and one or more compounds, extracts and the like have been found, the effect of such compounds can be studied in the laboratory in a more detailed fashion. Thus, inhibition or activation of the polypeptide according to the invention in vitro can be assayed again as a first step in order to subsequently assay the activity of the compound on the target organism, in this case one or more plant-pathogenic fungi. If appropriate, the compound can then be used as starting point for the further search and development of fungicidal compounds which are based on the original structure, but are optimized with regard to, for example, activity, toxicity or selectivity.

In order to find modulators, it is possible, for example, to incubate a synthetic reaction mix (e.g. in vitro transcription products) or a cellular component such as a membrane, a compartment or any other preparation comprising the polypeptides of the invention, together with a labeled or unlabeled substrate or ligand of the polypeptides in the presence and absence of a candidate molecule. The ability of the candidate molecule to inhibit the enzymic activity of the polypeptides of the invention is discernible, for example, by way of reduced binding of the labeled or unlabeled ligand or by way of reduced conversion of the labeled or unlabeled substrate. Molecules which inhibit the biological activity of the polypeptides of the invention are good antagonists and inhibitors.

Detection of the biological activity of the polypeptides of the invention may be improved by a “reporter system”. In this respect, reporter systems comprise, but are not limited to, calorimetrically or fluorimetrically detectable substrates which are converted into a product or a reporter gene which responds to changes in activity or expression of the polypeptides of the invention, or other known binding assays.

Another example of a method by which modulators of the polypeptides of the invention can be found is a displacement assay in which the polypeptides of the invention and a potential modulator are combined under suitable conditions with a molecule which is known to bind to said polypeptides of the invention, such as a natural substrate or ligand or a substrate or ligand mimetic. The polypeptides of the invention can themselves be labeled, for example fluorimetrically or colorimetrically, so that the number of polypeptides bound to a ligand or converted can be determined accurately. However, binding may also be monitored by means of the labeled or unlabeled substrate, ligand or substrate analog. Antagonist efficacy can be gauged in this way.

Effects such as cell toxicity are usually ignored in these in vitro systems. The assay systems test not only inhibitory or suppressive effects of the substances, but also stimulatory effects. The efficacy of a substance may be tested using concentration-dependent test series. Control mixtures without test substances or without enzyme may be used for evaluating said effects.

The host cells containing nucleic acids coding for an IPP isomerase of the invention, which are available on the basis of the present invention, also enable cell-based assay systems for identifying substances which modulate the activity of the polypeptides of the invention to be developed.

The modulators to be identified are preferably small organochemical compounds rather than the natural inhibitors of the enzyme, such as, for example, ligands of the enzyme or substrate analogs, inorganic or unspecific inhibitors which generally destroy or reduce the activity of an enzyme, for example by interfering in an unspecific manner with the protein structure or by reacting with reactive amino acids of the protein.

A method for identifying a compound which modulates the activity of an IPP isomerase from fungi and which can be used as fungicide in crop protection accordingly preferably comprises
a) contacting a polypeptide of the invention or a host cell containing said polypeptide with a chemical compound or with a mixture of chemical compounds under conditions which allow a chemical compound to interact with said polypeptide,
b) comparing the activity of the polypeptide of the invention in the absence of a chemical compound with the activity of the polypeptide of the invention in the presence of a chemical compound or of a mixture of chemical compounds, and
c) selecting the chemical compound which specifically modulates, preferably inhibits, the activity of the polypeptide of the invention, and, where appropriate,
d) testing the fungicidal action of the selected compound in vivo.

Particular preference is given here to determining the compound which specifically inhibits the activity of the polypeptide of the invention. The term “activity”, as used herein, refers to the biological activity of the polypeptide of the invention.

In one embodiment which follows a known assay for determining IPP isomerase, the IPP isomerase-catalyzed reaction is coupled to the reaction of isopentenyl transferase. The latter enzyme catalyzes conversion of dimethylallyl pyrophosphate and AMP to isopentenyladenine and pyrophosphate. Said pyrophosphate is further degraded by the enzyme pyrophosphatase. Phosphate produced in the process can be determined using a malachite green assay known to the skilled worker. The experimental approach can be depicted diagrammatically as follows:

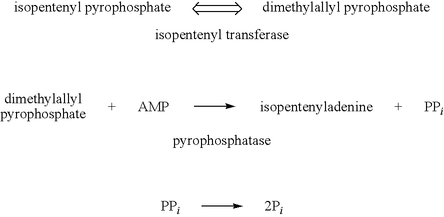

The enzymic activity of IPP isomerase or inhibition of said enzymic activity by an inhibitor is then measured based on the phosphate concentration. This involves monitoring the lower or inhibited activity of the polypeptide of the invention on the basis of the lower phosphate concentration in relation to a control mixture.

In the course of the present invention, the malachite green detection reagent was surprisingly found to be able to release and detect phosphate directly from the product but not from the substrate of IPP isomerase. As a result, in a particularly preferred embodiment of the described method, both isopentyl transferase and pyrophosphatase (IPPase) can be dispensed with.

Further possibilities of determining the enzymic activity of IPP isomerase are described inter alia also in Ramos-Valdivia (1997) and are expressly intended to be part of the present application.

The measurement may also be carried out in formats more commonly used for HTS or UHTS assays, for example in microtiter plates into which, for example, a total volume of from 5 to 50 μl per mixture or per well are introduced. The compound (candidate molecule) to be tested which potentially inhibits or activates the activity of the enzyme is introduced, for example, at a suitable concentration in assay buffer. The polypeptide of the invention is then added in the abovementioned assay buffer, thereby starting the reaction. The mixture is then incubated at a suitable temperature, and for example the concentration of the pyrophosphate produced is measured.

A further measurement is carried out in a corresponding mixture but without addition of a candidate molecule and without addition of a polypeptide of the invention (negative control). Another measurement is carried out in turn in the absence of a candidate molecule but in the presence of the polypeptide of the invention (positive control). Negative and positive controls therefore provide the comparative values for the mixtures in the presence of a candidate molecule.

In this way it was possible to identify inhibitors of IPP isomerase using the method of the invention.

In addition to the abovementioned methods of determining the enzymic activity of an IPP isomerase or inhibition of said activity and of identifying fungicides, other methods or inhibition assays, for example those which are already known, can, of course, also be used as long as said methods allow an IPP isomerase activity to be determined and inhibition of said activity to be detected by a candidate compound.

It was also found within the framework of the present invention that the inhibitors of an IPP isomerase of the invention, which were identified with the aid of a method of the invention, are useful for damaging or killing fungi in a suitable formulation.

For this purpose, a solution of the active compound to be tested may be pipetted, for example, into the cavities of microtiter plates. After the solvent has evaporated, medium is added to each cavity. The medium is treated beforehand with a suitable concentration of spores or mycelium of the fungus to be tested. The resulting concentrations of the active compound are, for example, 0.1, 1, 10 and 100 ppm.

The plates are subsequently incubated on a shaker at a temperature of 22° C., until sufficient growth can be established in the untreated control.

Evaluation is carried out photometrically at a wavelength of 620 nm. The active compound dosage which leads to 50% inhibition of fungal growth over the untreated control (ED₅₀) can be determined from the readings of the different concentrations.

The present invention therefore also relates to the use of modulators of IPP isomerase from fungi, preferably from plant-pathogenic fungi, as fungicides, and to methods of controlling preferably plant-pathogenic fungi, characterized in that an effective amount of a modulator, preferably an inhibitor, of an IPP isomerase is contacted with the fungus in question and/or its environment.

The present invention also relates to fungicides which have been identified with the aid of a method of the invention.

The present invention therefore likewise relates to methods for identifying fungicides and to the use of inhibitors of IPP isomerase from fungi, preferably from plant-pathogenic fungi, as fungicides. However, this should not include natural inhibitors such as analogs of the substrate of IPP isomerase, or analogs of the transitional state of the substrate, and unspecific inhibitors which exhibit a clear inhibitory action also with enzymes other than IPP isomerase or which have a fundamental inhibitory action due to damage of the protein structure, as well as inorganic compounds. A specific inhibitor should have an inhibitory action on IPP isomerase which is greater than said action on a different enzyme by a factor of at least 10, preferably 20, particularly preferably 50 and preferentially 100.

The present invention also relates to fungicides which have been identified with the aid of a method according to the invention.

Compounds which are identified with the aid of a method according to the invention and which, owing to inhibition of the fungal IPP isomerase, are fungicidally active can thus be used for the preparation of fungicidal compositions.

Depending on their respective physical and/or chemical characteristics, the active compounds which have been identified can be converted into the customary formulations, such as solutions, emulsions, suspensions, powders, foams, pastes, granules, aerosols, very fine capsules in polymeric substances and in coating compositions for seed and also ULV cold- and hot-fogging formulations.

These formulations are produced in a known manner, for example by mixing the active compounds with extenders, that is, liquid solvents, liquefied gases under pressure, and/or solid carriers, optionally with the use of surfactants, that is, emulsifiers and/or dispersants and/or foam-formers. In the case of the use of water as an extender, organic solvents can, for example, also be used as cosolvents. As liquid solvents, there are suitable in the main: aromatics, such as xylene, toluene or alkylnaphthalenes, chlorinated aromatics or chlorinated aliphatic hydrocarbons, such as chlorobenzenes, chloroethylenes or methylene chloride, aliphatic hydrocarbons, such as cyclohexane or paraffins, for example mineral oil fractions, alcohols, such as butanol or glycol as well as their ethers and esters, ketones, such as acetone, methyl ethyl ketone, methyl isobutyl ketone or cyclohexanone, strongly polar solvents, such as dimethylformamide and dimethyl sulfoxide, and water. By liquefied gaseous extenders or carriers are meant liquids which are gaseous at ambient temperature and under atmospheric pressure, for example aerosol propellants, such as halogenohydrocarbons and butane, propane, nitrogen and carbon dioxide. As solid carriers there are suitable: for example ground natural minerals, such as kaolins, clays, talc, chalk, quartz, attapulgite, montmorillonite or diatomaceous earth, and ground synthetic minerals, such as highly disperse silica, alumina and silicates. As solid carriers for granules there are suitable: for example crushed and fractionated natural rocks such as calcite, marble, pumice, sepiolite and dolomite, as well as synthetic granules of inorganic and organic meals, and granules of organic material such as sawdust, coconut shells, maize cobs and tobacco stalks. As emulsifiers and/or foam-formers there are suitable: for example nonionic and anionic emulsifiers, such as polyoxyethylene fatty acid esters, polyoxyethylene fatty alcohol ethers, for example alkylaryl polyglycol ethers, alkylsulfonates, alkyl sulfates, arylsulfonates and protein hydrolysates. As dispersants there are suitable: for example lignin-sulfite waste liquors and methylcellulose.

Adhesives such as carboxymethylcellulose and natural and synthetic polymers in the form of powders, granules or latices, such as gum arabic, polyvinyl alcohol and polyvinyl acetate, as well as natural phospholipids, such as cephalins and lecithins, and synthetic phospholipids can be used in the formulations. Further additives may be mineral and vegetable oils.

It is possible to use colorants such as inorganic pigments, for example iron oxide, titanium oxide and Prussian Blue, and organic dyestuffs, such as alizarin dyestuffs, azo dyestuffs and metal phthalocyanine dyestuffs, and trace nutrients such as salts of iron, manganese, boron, copper, cobalt, molybdenum and zinc.

The formulations generally comprise between 0.1 and 95 percent by weight of active compound, preferably between 0.5 and 90%.

The active compounds according to the invention, as such or in their formulations, can also be used as a mixture with known fungicides, bactericides, acaricides, nematicides or insecticides, for example in order to widen in this way the spectrum of action or to prevent the build-up of resistance. In many cases, synergistic effects are achieved, i.e. the efficacy of the mixture exceeds the efficacy of the individual components.

When employing the compounds according to the invention as fungicides, the application rates can be varied within substantial ranges, depending on the application.

All plants and plant parts may be treated in accordance with the invention. In the present context, plants are understood as meaning all plants and plant populations, such as desired and undesired wild plants or crop plants (including naturally occurring crop plants). Crop plants may be plants which can be obtained by traditional breeding and optimization methods or by biotechnological and recombinant methods or combinations of these methods, including the transgenic plants and including those plant varieties which are capable, or not capable, of protection by Plant Breeders' Rights. Plant parts are to be understood as meaning all aerial and subterranean parts and organs of the plants, such as shoot, leaf, flower and root, examples which are mentioned being leaves, needles, stems, stalks, flowers, fruiting bodies, fruits and seeds, and also roots, tubers and rhizomes. The plant parts also include harvested material and vegetative and generative propagation material, for example cuttings, tubers, rhizomes, slips and seeds.

The treatment according to the invention of the plants and plant parts with the active compounds is effected directly or by acting on their environment, habitat or store by the customary treatment methods, for example by dipping, spraying, vaporizing, fogging, scattering, brushing on and, in the case of propagation material, in particular seeds, furthermore by coating with one or more coats.

The examples which follow illustrate various aspects of the present invention and are not to be construed as limiting.

### EXAMPLES

### Example 1

### Cloning, Expression and Purification of U. maydis IPI1

For heterologous expression of the ipi1 gene, said gene was amplified with the gene-specific oligonucleotides Idi-c (5′-CTCGAGGATCCAGGAGGCGGTGAATG-3′) and Idi-n (5′-CTCGCATATGTCGACCGCCACCGTCAC-3′) by means of PCR and inserted via the introduced NdeI and BamHI cleavage sites in the pET21b vector (Novagen). The plasmids obtained were transformed into the E. coli BL21 (DE3) strain.

5 ml of selection medium (dYT medium containing 100 μg/ml ampicillin) were inoculated with a single colony and incubated on a shaker at 37° C. overnight. A glycerol stock culture was prepared as follows: mix 900 μl of culture and 100 μl of sterile glycerol and freeze at −70° C. A preculture was prepared by inoculating 12 ml of dYT medium with 25 μl of the stock culture and incubating the culture on a shaker at 37° C. overnight. The main culture was inoculated 1:40, i.e. 12 ml of preculture plus 500 ml of dYT medium+100 μg/ml ampicillin. The culture was grown at 37° C. with shaking and, after reaching 0.8 OD₆₀₀, induced by adding 1 mM IPTG (final concentration). After 5 hours of incubation at 37° C., the cells were harvested by centrifugation and the pellet was frozen at −70° C.

The cell pellet of a 500 ml expression culture was resuspended in 35 ml of lysis buffer (50 mM Tris, 1% glycerol, 1 mM DTT, 300 mM NaCl, 0.5% Tween 20, pH 7.5). The cells were disrupted on ice using a sonicator, sonicating for 8 times 45 seconds with 45 second intervals. The soluble and insoluble fractions were separated by centrifugation (30 min at 4° C. and 10 000 rpm). The supernatant was bound to a 50% Ni-NTA-agarose matrix from Qiagen at 4° C. for 60 minutes and transferred to an empty column. The binding capacity of said Ni-NTA matrix is 9 mg of protein per 1 ml of agarose. The column was washed twice with in each case 44 ml of lysis buffer+10 mM imidazole. Elution was carried out in 2 ml fraction steps with lysis buffer+250 mM imidazole. The fractions containing the purified enzyme were then pooled and diluted to 1 mg/ml. Glycerol was added to a final concentration of 10%. The enzyme was stored at −70° C.

### Example 2

### Identification of IPP Isomerase Modulators in an Inhibition Assay

The test was carried out in 384-well MTPs from Greiner (transparent). The negative control omitted the enzyme. 5 μl of R1 buffer (10 mM Tris/HCl pH 7.5, 20 mM MgCl₂, 10% glycerol) or the substance to be tested ( 1/10 of assay volume) were incubated together with 20 μl of substrate solution (0.15 mM IPP in reaction buffer R1), 25 μl of enzyme mix (0.59 μg/ml purified protein (IPP isomerase) in reaction buffer R1) at 37° C. for 25 minutes. Malachite green staining solution (50 μl) was added, followed by incubation at RT for 90 minutes. A change in absorbance was detected at 620 nm.

### Example 3

### Detection of Fungicidal Action of the Identified Inhibitors of IPP Isomerase

A methanolic solution of the active compound identified on the basis of a method of the invention (example 3), to which an emulsifier has been added, is pipetted into the cavities of microtiter plates. After the solvent has evaporated, 200 μl of potato-dextrose medium are added to each cavity. The medium is treated beforehand with suitable concentrations of spores or mycelia of the fungus to be tested.

The resulting concentrations of the active compound are 0.1, 1, 10 and 100 ppm. The resulting concentration of the emulsifier is 300 ppm.

The plates are subsequently incubated on a shaker at a temperature of 22° C., until sufficient growth can be established in the untreated control. Evaluation is carried out photometrically at a wavelength of 620 nm. The active compound dosage resulting in 50% inhibition of fungal growth over the untreated control (ED₅₀) is calculated from the readings of the different concentrations.

### REFERENCES

Cheng, F. and Oldfield, E. (2004): Inhibition of Isoprene Biosynthesis Pathway Enzymes by Phosphonates, Bisphosphonates, and Diphosphonates. J. Med. Chem. 47, 5149-5158.
Mayer, M. P., F. M. Hahn, D. J. Stillman & C. D. Poulter (1992): Disruption and mapping of IDI, the gene for the isopentenyl diphosphate isomerase in Saccharomyces cerevisiae. Yeast. 8, 743-748.
Ramos-Valdivia A., van der Heijden, R. and Verpoorte, R. (1997): Isopentenyl diphosphate isomerase: a core enzyme in isoprenoid biosynthesis. A review of ist biochemistry and function. Nat. Prod. Rep. 14, 591-603.
Rohdich F., Bacher A. and Eisenreich W. (2004): Perspectives in anti-infective drug design. Bioorganic Chemistry 32, 292-308.
Street et al. (1994): Identification of Cys139 and Glu207 as catalytically important groups in the active site of isopentenyl diphosphate:dimethylallyl diphosphate isomerase. Biochemistry 33, 4212-4217.
Thompson K., Dunford J. E., Ebetino F. H., Rogers M. J. (2002): Identification of a bisphosphonate that inhibits isopentenyl diphosphate isomerase and farnesyl diphosphate synthase. Biochemical and Biophysical Research Communications 290, 869-873.
Wouters J., Oudjama Y., Barkley S. J., Tricot C., Stalon V., Droogmans L., Poulter C. D. (2003): Catalytic mechanism of Escherichia coli isopentenyl diphosphate isomerase involves Cys-67, Glu-116, and Tyr-104 as suggested by crystal structures of complexes with transition state analogues and irreversible inhibitors. J. Biol. Chem. 278, 11903-1198.

1. A method for identifying fungicides, wherein
(a) a fungal polypeptide having the enzymic activity of an IPP isomerase is contacted with a chemical compound or a mixture of chemical compounds under conditions which allow said chemical compound to interact with said polypeptide,
(b) the activity of said IPP isomerase in the absence of a chemical compound is compared with the activity of said IPP isomerase in the presence of a chemical compound or of a mixture of chemical compounds, and
(c) the chemical compound which specifically inhibits said IPP isomerase is selected.

2. The method as claimed in claim 1, the IPP isomerase activity is determined by measuring the generation of phosphate from the product of the reaction catalyzed by said IPP isomerase.

3. The method as claimed in claim 1, wherein an inhibition of the enzymic activity of the IPP isomerase in the presence of a chemical compound is determined on the basis of a decreasing amount of phosphate.

4. The method as claimed in claim 1, wherein the IPP isomerase reaction is determined directly by a malachite green assay.

5. The method as claimed in claim 1, wherein, in a further step (d), the fungicidal action of the identified compound is assayed by contacting said compound with a fungus.

6. The method as claimed in claim 1, wherein an IPP isomerase from a plant-pathogenic fungus is used.

7. A method for identifying fungicide comprising using a polypeptide having the activity of an IPP isomerase for identifying fungicides.

8. A fungicide comprising an inhibitor of a polypeptide having the activity of an IPP isomerase.

9. A method for controlling plant-pathogenic fungi, wherein
(a) a fungicidal compound is identified in a method as claimed in claim 1,
(b) the identified compound is formulated in a suitable way, and
(c) contacted with the plant-pathogenic fungus and/or an environment thereof.

10. A fungicidal compound for preparing a fungicidal agent, said compound being found by a method as claimed in claim 1.

11. A nucleic acid comprising a sequence selected from the group consisting of:
(a) a sequence according to SEQ ID NO: 1,
(b) sequences coding for a polypeptide comprising the amino acid sequence according to SEQ ID NO: 2,
(c) sequences which hybridize to the sequences defined under a) at a hybridization temperature of 42-65° C., and
(d) sequences which are at least 80% identical to the sequences defined under a) and b).

12. A DNA construct comprising a nucleic acid as claimed in claim 11 and a heterologous promoter.

13. A vector comprising a nucleic acid as claimed in claim 11, or a DNA construct thereof.

14. The vector as claimed in claim 13, wherein the nucleic acid is functionally linked to regulatory sequences which ensure expression of said nucleic acid in pro- or eukaryotic cells.

15. A host cell comprising a nucleic acid as claimed in claim 11, a DNA construct thereof and/or a vector thereof.

16. A polypeptide having the biological activity of an IPP isomerase, which is encoded by a nucleic acid as claimed in claim 11.

17. A polypeptide having the biological activity of an IPP isomerase, which comprises an amino acid sequence according to SEQ ID NO: 2.

18. A nucleic acid according to claim 11 that comprises a sequence that is at least 85% identical to the sequences of
(a) a sequence according to SEQ ID NO: 1, and/or
(b) sequences coding for a polypeptide comprising the amino acid sequence according to SEQ ID NO: 2.

19. A nucleic acid according to claim 11 that comprises a sequence that is at least 90% identical to the sequence of
(a) a sequence according to SEQ ID NO: 1, and/or
(b) sequences coding for a polypeptide comprising the amino acid sequence according to SEQ ID NO: 2.


SEQUENCE LISTING (2 sequences)

SEQ ID NO: 1 - DNA, 789 bp, Ustilago maydis
  CDS (1)..(789)
atgtcgaccgccaccgtcaccgagacagcgacacatcgcacctcgtccaccatcacgctagactcagcgccactcaccggctatgatgaggagcagatccgtctcatggaagagcgatgtatcgtgctcgacaatgacgacaagtacgtccgcgatggcagtaagaaggaatgccatctcatgaccaacatcaacaagggtctcttgcatcgtgctttctccgtgttcctctttgatcctactacgggaaagctgttgcttcagcgaagggcgctcgaaaagatcactttccccaacatgtggaccaacacttgctgctctcatccgttggcgatcaagggagagctcgaggaagcagagcagatcggtgtacgccgtgcagcgcagcgcaagctcgatcacgaactcggcatccgcgccgaacaagtgccattggatgaattccagtacctcacccgaatccactacctcgctcccaacaacgacgccaataacatgtggggcgaacacgaaatcgactacatcctcttcatcactgccgacgtcacgttgaaacccaacctgaacgaggtctgcgataccaaatgggtttcgcccgaagagttgaaggcgctcatgaccgagttggaccctgcgtcatttacgccatggttcaagttgattgtgcacaagtttctcttcccctggtggagcgagttgctcgcaaggagaggcgcggatcacagcaaaccatttgacgccaagtcgctgtcggatcttacagatcacagcattcaccgcctcctctga

SEQ ID NO: 2 - protein, 262 aa, Ustilago maydis
MSTATVTETATHRTSSTITLDSAPLTGYDEEQIRLMEERCIVLDNDDKYVRDGSKKECHLMTNINKGLLHRAFSVFLFDPTTGKLLLQRRALEKITFPNMWTNTCCSHPLAIKGELEEAEQIGVRRAAQRKLDHELGIRAEQVPLDEFQYLTRIHYLAPNNDANNMWGEHEIDYILFITADVTLKPNLNEVCDTKWVSPEELKALMTELDPASFTPWFKLIVHKFLFPWWSELLARRGADHSKPFDAKSLSDLTDHSIHRLL